(19) United States
(12) Patent Application Publication
(10) Pub. No.: US 2024/0335652 A1
(43) Pub. Date: Oct. 10, 2024

(54) AUTOLOGOUS LEFT VENTRICLE ASSIST DEVICE

(71) Applicant: Sean Boutros, M.D., F.A.C.S., Houston, TX (US)

(72) Inventor: Sean Boutros, M.D., F.A.C.S., Houston, TX (US)

(21) Appl. No.: 18/627,983

(22) Filed: Apr. 5, 2024

Related U.S. Application Data
(60) Provisional application No. 63/457,466, filed on Apr. 6, 2023.

(51) Int. Cl. A61M 60/882; A61M 60/178; A61M 60/268; A61M 60/424; A61M 60/837; A61M 60/857; A61N 1/36

(52) U.S. Cl. CPC A61M 60/882; A61M 60/178; A61M 60/268; A61M 60/424; A61M 60/837; A61M 60/857; A61M 2205/054; A61N 1/36003

(57) ABSTRACT

An autologous left ventricular assist device (ALVAD) is implanted into a skeletal muscle of a patient, such as being nested in the Latissimus Dorsi muscle. This implantation may be referred to as a training or prefabrication step and may last for 3 to 6 months or even longer. The ALVAD may include a plurality of ribs distributed in a spiral configuration as structural support and may include a plurality of electrodes operative to cause repeated contractions of the latissimus dorsi muscle. Once trained, the autologous LVAD—now integrated into the conditioned muscle—may be repositioned, reshaped and, specifically, moved into proximity with the patient's own heart and operatively coupled in fluid communication therewith for repeatedly contracting so as to pump oxygenated blood to and through the aorta and to the patient's organs and tissues.

### REFERENCE TO RELATED APPLICATIONS

This is a non-provisional patent application claiming the priority of provisional application 63/457,466 filed Apr. 6, 2023 titled Autologous Left Ventricle Assist Device, which is incorporated herein by reference.

### BACKGROUND OF THE INVENTION

This invention relates generally to ventricular assist devices and, more particularly, to a prefabricated partially autologous left ventricular assist device.

The organs of a human body require oxygen and oxygen is delivered to the organs via oxygenated blood. Arteries carry oxygenated blood to a person's organs and then veins carry the deoxygenated blood back to the lungs which add oxygen to the blood and return the oxygen-rich blood to the heart.

Congestive heart failure is a complex condition where output of the heart is insufficient to meet demands. More particularly, it means that the heart muscle has become less able to contract over time or has a mechanical problem that limits its ability to fill with blood. As a result, it can't keep up with the body's demand, and blood returns to the heart faster than it can be pumped out. Thus, it becomes congested, or backed up. This pumping problem means that not enough oxygen-rich blood can get to the body's other organs. In this situation, a ventricular assist device (VAD) may be surgically implanted to pump blood more efficiently or simply to assist the body's own pumping action.

Specifically, a ventricular assist device (VAD) is a device that helps pump blood from the lower chambers (ventricles) of a person's heart to the rest of his body. A VAD may be implanted in one or both ventricles of a patient's heart or the main vessels leaving the ventricle, such as by surgery, and, if placed in the left ventricle or the aorta is called a left ventricular assist device (LVAD). The left ventricle chamber of the heart receives oxygenated blood and then pumps the same through the aorta to organs of the body. A VAD may be useful to pump blood while a patient waits for a heart transplant or simply to efficiently pump a patient's blood long-term or permanently. Unfortunately, a ventricular assist device has its own shortcomings.

Although presumably effective for their intended purposes, traditional LVADs require power, such as to operate a pump, and may fail—potentially causing immediate heart failure or even death. Therefore, it would be desirable to have method and apparatus that utilizes a patient's own tissues to form an LVAD that does not require any artificial or external motorized pump or electrical power in order to pump the blood. In fact, the term autologous, by definition, means “taken from one's own tissues, cells, or DNA.” (National Cancer Institute, https://www.cancer.gov/publications/dictionaries/cancer-terms/def/autologous)

Therefore, it would be desirable to have a partially autologous left ventricular assist device that includes a patient's skeletal muscle such as the Latissimus Dorsi muscle (often referred to as the “lats,”) that has been trained to pulsate in the manner of the heart muscle in order to become an autologous left ventricular assist device (ALVAD). Further, it would be desirable to have an ALVAD having a plurality of spiral ribs and electrodes configured to train a patient's own skeletal muscle to twitch/contract in a regular and repetitious manner that will pump blood to and through the aorta with enhanced pressure.

### SUMMARY OF THE INVENTION

Therefore, a general object of this invention is to provide an autologous left ventricular assist device (ALVAD) that may be implanted in a skeletal muscle of the patient's body, such as in a latissimus dorsi muscle, for integration and prefabrication therewith over the course of a plurality of calendar weeks,

Another object of this invention is to provide an autologous left ventricular assist device as aforesaid, that may include a plurality of ribs arranged in a spiral configuration to allow for compliance of the device to allow for diastolic filling of the device.

Another objective of this invention is to provide a removal internal surface of the device to induce a capsule formation to act as a pseudo endothelium of the device to allow for a smooth surface to interface with the blood.

A further object of this invention is to provide an ALVAD, as aforesaid, that includes a plurality of electrodes configured to cause a contraction when energized.

Another object of this invention is to provide a hybrid and prefabricated ALVAD that includes the original implanted ALVAD and portions of the latissimus dorsi muscle that have become integrated with the implanted ALVAD.

Still another object of this invention is to provide an autologous left ventricular assist device, as aforesaid, in which the primary conduit may be the patient's own pulmonary vein or left ventricle or aorta.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

FIG. 1 is a perspective view of an autologous left ventricle assist device according to the present invention, illustrated implanted in a latissimus dorsi muscle of a patient;

FIG. 2 is a perspective view of the assist device as in FIG. 1 in fluid communication with a human heart or alternatively directly to the aorta;

FIG. 2A is a sectional view taken along line 2A-2A of FIG. 2;

FIG. 3 is another perspective view of the assist device as an FIG. 1, illustrated on an isolated and enlarged view;

FIG. 4 is another perspective view of the assist device as in FIG. 1, illustrated with a portion of the latissimus dorsi muscle having integrated itself to the assist device implanted therein;

FIG. 5 is a top view of the assist device as in FIG. 3; and

FIG. 6 is a sectional view taken along line 6-6 of FIG. 5.

### DESCRIPTION OF THE PREFERRED EMBODIMENT

A method and apparatus for assisting according to a preferred embodiment of the present invention will now be described with reference to the accompanying drawings.

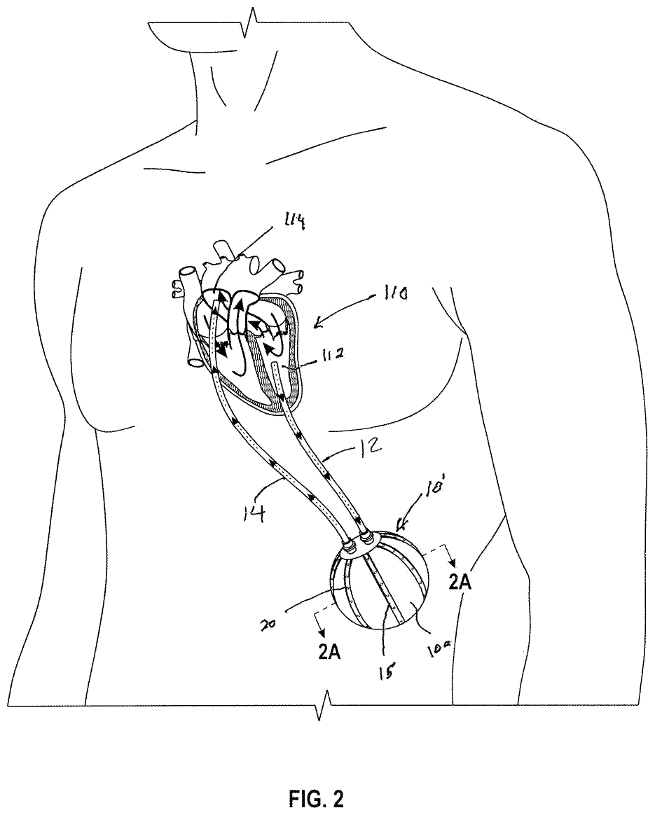

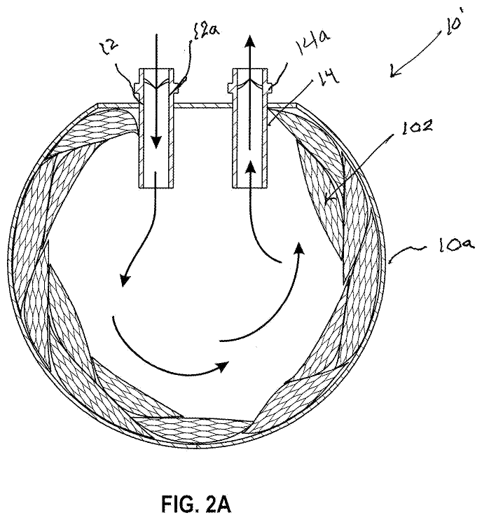

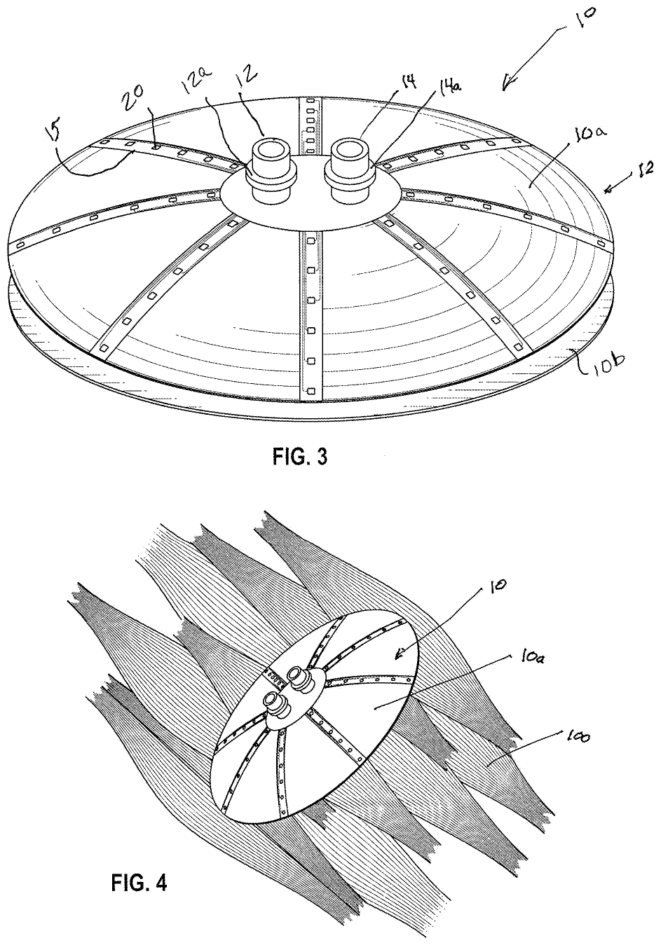

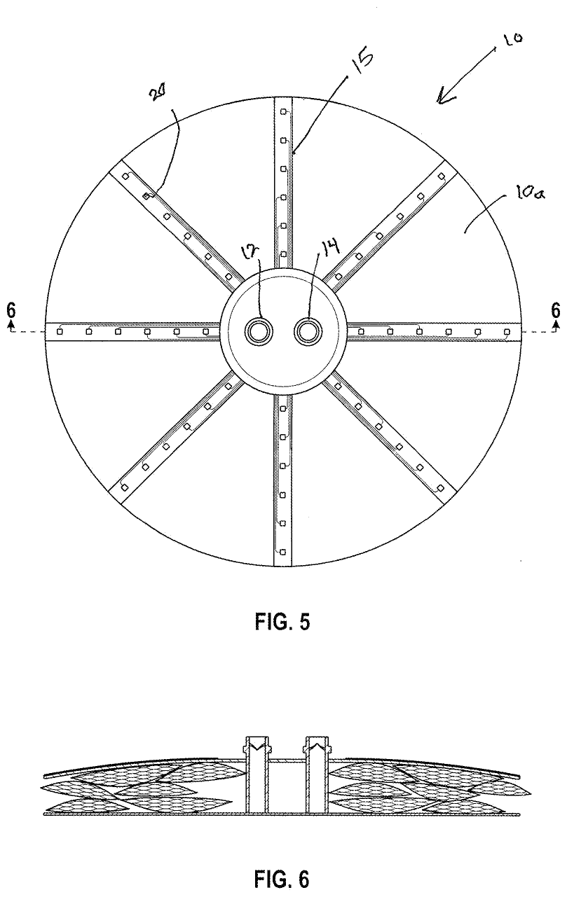

Accordingly, an autologous left ventricular assist device 10 (ALVAD) according to the present invention is implanted into a skeletal muscle of a person such as being nested in the Latissimus Dorsi muscle 100. This implantation may be referred to as a training or prefabrication step and may last for 3 to 6 months or even longer. The ALVAD 10 may include a plurality of ribs distributed in a spiral configuration as structural support and may include a plurality of electrodes 20 operative to cause repeated contractions of the latissimus dorsi muscle 100. Although not shown, it is understood that the ALVAD may be connected to a power source such as a battery with wires or may be wireless during the time that it is implanted in the patient's muscle that is being trained. Once trained, the autologous LVAD 10—now integrated into the conditioned muscle 100 (FIG. 2)—may be repositioned, reshaped and, specifically, moved into proximity with the patient's own heart 110 (FIG. 2) and operatively coupled thereto (i.e., in fluid communication) for repeatedly contracting so as to pump oxygenated blood to and through the aorta 114 and to the patient's organs and tissues. In other words, a patient's own muscles can be used to assist the heart and pumping blood while perhaps waiting on a heart transplant, surgery, or the like.

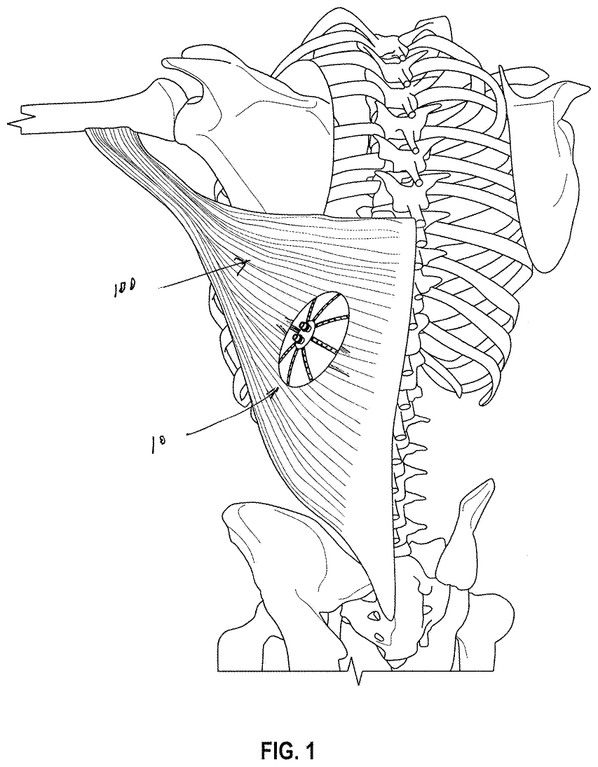

The ALVAD 10 may be constructed of a substrate referred to later as a training member or training device that is durable yet semi-flexible for implantation inside a human body while remaining flexible for intended contractions. The training member includes a body 12 or housing constructed of a material that will integrate itself with the fibers of a patient's muscle into which it is implanted (FIG. 1). In this regard, the ALVAD may initially include a mesh or webbing layer (not shown) to which human tissue will readily adhere. Similar to that of an artificial heart, the ALVAD 10 may be constructed using bioplastics and polymers, including polyurethane, which is both durable and semi-flexible. Specifically, the ALVAD may include an outer layer 10a constructed of the semi-flexible material and an inner layer 10b constructed of silicone.

In a critical aspect, the ALVAD may include a plurality of ribs 15 and electrodes 20 coupled to an outer surface of the outer layer 10a, the electrodes being configured to cause the substrate to twitch or contract when energized. In an embodiment, the ribs 15 may be arranged in a spiral or other configuration. It is understood that the spiral ribs 15 provide structure and prevent the ALVAD from collapsing when subjected to the repeated contractions. In addition, the plurality of electrodes 20 may be hardwired using bio electronics although enhancements in electrode technology may allow wireless signal transmission and receipt. Stated summarily, the plurality of electrodes 20 may be intermittently energized to stimulate the latissimus dorsi muscle 100 in which the ALVAD training device 12 is implanted. In fact, the latissimus dorsi muscle 100 may be trained to contract with the same rhythm of the human heart 110 as will be described later.

Further the ALVAD may contain an inner layer 10b of silicone of similar material. This internal layer may allow for formation of a capsule. This capsule would allow for a smooth surface that would interface with the patient's blood. This capsule would also assist in manipulation of the device to allow for reshaping from a flat layer into a compliant sphere 10′ (FIG. 2) to receive blood that would then be pumped. This inner layer 10b may be removable and actually removed while the device, along with the integrated muscle 100 is reshaped into the compliant sphere 10′ and contracting vessel. The muscle is essentially folded and is referred to with reference character 102.

Further the ALVAD, when it has become sufficiently integrated into the latissimus dorsi muscle 100, will twitch or contract along with the ALVAD 10, i.e., when the electrodes 20 are energized. Again, the preferred methodology is a muscle training and strengthening process that repeatedly fatigues the latissimus dorsi muscle 100 so that it will repeatedly grow back stronger in a manner that is analogous to the training process in which a human person exercises muscles during weight training by fatiguing one's muscles which then causes them to grow back stronger and stronger. In this manner, the ALVAD may be referred to as a training device which will eventually be transplanted to a position proximate a patient's heart 115 and operatively coupled thereto. More particularly, when the muscle has been sufficiently trained, the modified or enhanced ALVAD includes the originally implanted unit along with the portion of the muscle that has been integrated, conditioned, and a functional part of the ALVAD itself (see FIG. 4). Stated another way, when the training device is sufficiently trained (such as being trained to repeatedly contract and relax), the training device will be deemed to have transformed into an autologous pumping device. It is understood that the autologous pumping device has all of the same structures and attributes as previously described above relative to being a training device.

In a critical aspect, the ALVAD may include two openings 12, 14 that would penetrate the muscle to allow for eventual placement of rings 12a, 14a, i.e. valves configured to communicate with respective tubes that will be attached later in order to control the direction of blood flow from and to the heart. Stated another way, these openings may include a ring to allow for eventual attachment of a valve and cannulas/conduits. Preferably, the primary conduit 12 may be connected to the ascending aortic chamber for directing blood into the ALVAD 10. By definition, a cannula is a thin tube that medical professionals insert into a patient cavity, such as their nose, a vein, or the like. Medical personnel use cannulas to drain fluid, administer medication or to provide oxygen, etc. In the present invention, cannulas are mounted and positioned for fluid communication between a patient's heart and the ALVAD.

In an embodiment, the primary cannula/conduit 16 may actually be the patient's own pulmonary vein bringing oxygenated blood from the lungs to the heart or, in an embodiment, merely attached thereto. For context, the pulmonary veins are blood vessels that transfer freshly oxygenated blood from the lungs to the left atria of the heart. These veins are part of the pulmonary circulation system or circuit. By contrast, pulmonary arteries transfer oxygen-poor blood from the heart to the lungs to be re-oxygenated. The conduit may also be the patient's left ventricle 112 or aorta 114. Then, a twitch or pump of the modified/trained ALVAD 10 causes the received blood to flow with enhanced speed or force through the secondary conduit 14 back to the aorta 114 (i.e., preferably above the ascending aorta) and to organs of the patient's body (FIG. 2).

Further, the rings may be either temporary or permanent. There could be an outer ring surrounding both rings to create an annulus that would provide semi-rigid support to both rings in order to maintain the internal volume of the ALVAD. Specifically, each ring may be referred to as a support ring and each ring may be constructed using stainless steel or medical grade hard plastic so as to prevent a collapse of the openings and to enhance fluid flow into and out of the interior area of the training device.

It is understood that while certain forms of this invention have been illustrated and described, it is not limited thereto except insofar as such limitations are included in the following claims and allowable functional equivalents thereof.

1. A method for repeatedly pumping oxygenated blood using an autologous left ventricle assist device (ALVAD) implanted in a patient, said method comprising:
surgically implanting a training member in a latissimus dorsi muscle of the patient for a medically predetermined amount of time, said training member having an external layer for promoting integration therewith,
said training member including a continuous wall defining an interior area and defining an inlet opening and an outlet opening displaced from the inlet opening such that said inlet and outlet openings are in communication with the interior area;
wherein said training member includes (1) a network of ribs coupled to said continuous wall each having a spiral configuration and (2) a plurality of electrodes distributed along said network of ribs;
repeatedly energizing and deenergizing said plurality of electrodes so as to repeatedly contract and relax said network of ribs, respectively, until said training member is transformed into an autologous pumping device; and
surgically repositioning, shaping and placing said autologous pumping device into fluid communication with the patient's vascular system for pumping oxygenated blood.

2. The method as in claim 1, further comprising:
a first cannula having a first end coupled to and in fluid communication with said inlet opening of said training device for directing blood into the interior area of said training device; and
a second cannula having a first end coupled to and in fluid communication with said outlet opening of said training device for directing blood out and away from the training device.

3. The method as in claim 2, further comprising:
a first support ring positioned in said inlet opening of said training device that is configured to promote fluid flow into said interior area of the training device; and
a second support ring positioned in said outlet opening of said training device that is configured to promote fluid flow out of the interior area of the training device.

4. The method as in claim 1, wherein said medically predetermined amount of time is at least 3 months.

5. An autologous left ventricle assist device (ALVAD) for pumping oxygenated blood through a pulmonary system when implanted in a patient, comprising:
a training member having an external layer configured to integrate with a latissimus dorsi muscle of the patient for a medically predetermined amount of time when surgically implanted therein,
said training member including a continuous wall defining an interior area and defining an inlet opening and an outlet opening displaced from said inlet opening, said inlet and outlet openings being in communication with said interior area,
said training member having (1) a network of ribs coupled to said continuous wall each having a nonlinear configuration and (2) a plurality of electrodes distributed along said network of ribs, said training member being configured to repeatedly contract and relax along said network of ribs when said plurality of electrodes are energized and deenergized, respectively, until said training member is transformed into an autologous pumping device;
wherein said autologous pumping device is operative to pump oxygenated blood when surgically interfaced in fluid communication with the patient's pulmonary system.

6. The autologous left ventricle assist device as in claim 5, further comprising:
a first cannula having a first end coupled to and in fluid communication with said inlet opening of said training device for directing blood into the interior area of said training device; and
a second cannula having a first end coupled to and in fluid communication with said outlet opening of said training device for directing blood out and away from the training device.

7. The autologous left ventricle assist device as in claim 5, further comprising:
a first support ring positioned in said inlet opening of said training device that is configured to promote fluid flow into said interior area of the training device; and
a second support ring positioned in said outlet opening of said training device that is configured to promote fluid flow out of the interior area of the training device.

8. The autologous left ventricle assist device as in claim 5, wherein said medically predetermined amount of time is at least 3 months.
